(19) United States
(12) Patent Application Publication
(10) Pub. No.: US 2017/0205322 A1
(43) Pub. Date: Jul. 20, 2017

(54) CENTRIFUGE CHAMBER

(71) Applicant: Cytori Therapeutics, Inc., San Diego, CA (US)

(72) Inventors: Douglas M. Arm, San Diego, CA (US); Raphael Shapiro, San Diego, CA (US); Olga Souverneva, San Diego, CA (US); Johnson Chung Sing Yu, San Diego, CA (US); Andrew Holman, San Diego, CA (US); Robert Shanahan, San Diego, CA (US)

(21) Appl. No.: 15/324,596

(22) PCT Filed: Jul. 6, 2015
(86) PCT No.: PCT/US2015/039250

§ 371 Date: Jan. 6, 2017

Related U.S. Application Data
(60) Provisional application No. 62/022,131, filed on Jul. 8, 2014.

(51) Int. Cl. G01N 1/40; C12N 5/071; C12N 5/077; B04B 7/08; B04B 5/04

(52) U.S. Cl. CPC G01N 1/4077; B04B 7/08; B04B 5/0442; C12N 5/0653; C12N 5/0669; C12N 5/069; G01N 2001/4083

(57) ABSTRACT

Provided herein is a centrifuge chamber, useful in the separation and concentration of particles from cell suspensions. In particular, the centrifuge chamber described herein can be advantageously used to separate and concentrate desired cell populations from cell suspensions, e.g., from biological samples or tissues. Also provided are methods of separating and concentrating desired particles (e.g., desired cell populations), from suspensions (e.g., cell suspensions) using the centrifuge chamber described herein. Kits including a centrifuge chamber as described herein, and optionally other components (e.g., a centrifuge), are also provided.

### STATEMENT REGARDING FEDERALLY SPONSORED R&D

The present invention was made with government support under the following grants: HHSθ100201200008C awarded by the Department of Health and Human Services. The government has certain rights in the invention.

### BACKGROUND

Fluids, such as biological fluids are suspensions and can be separated into their constituent parts or fractions. For example, digested adipose tissue contains a stromal cell component and an adipocyte component that can be separated based upon the different densities of different cells and other particles in suspension in a device such as a centrifuge.

Devices for the separation of biological fluids into their constituent parts that utilize centrifugation have been described. For example, U.S. Pat. Nos. 7,390,484, 7,571,115, 7,585,670, and 7,687,059 describe exemplary devices that are useful for the processing of adipose tissue and separation and concentration of regenerative cell populations therefrom. U.S. Patent Application Publication No. 2008/0014181 describes an apparatus for separation of cells from a tissue sample. The device described in U.S. Patent Application Publication No. 2008/0014181 includes a centrifuge bowl, having an inner chamber with two lobes that protrude on opposite sides of the center of the bowl. In operation, centrifugal forces “pack” the cells into the lobes, while undesired fluid and components flow down the walls of the bowl to the center of the bowl via gravity. After removal of the undesired fluid components from the bottom of the bowl, fluid is jetted towards the lobes via spray nozzle located in the center of the centrifuge bowl, in order to dislodge and break up the cell pellets. The dislodged, broken up cells then flow to the bottom of the centrifuge bowl via gravity, where they are subsequently harvested.

Cell yield and viability, sterility, and processing time are parameters of utmost importance in the context of isolation of cellular components from biological tissues or fluids, e.g. for downstream use in either clinical or research settings. The need for devices designed to maximize cell viability, sterility, processing time, and ease-of operation while minimizing the device footprint is manifest.

### SUMMARY

The embodiments described herein relate to herein are improved centrifuge chambers and methods of using the same, which include features that advantageously improve upon sterility, processing time, cell viability, usability and footprint of other separation devices.

### BRIEF DESCRIPTION OF THE DRAWINGS

FIG. 1A is a perspective view of an exemplary centrifuge chamber including a fluid transfer assembly as described herein.

FIG. 1B is an exploded view of the exemplary centrifuge chamber shown in FIG. 1A.

FIG. 1C is a side view of an exemplary centrifuge chamber (without a fluid transfer assembly) as described herein, wherein the right side of the centrifuge chamber is shown cutaway.

FIG. 1D is a cutaway section A-A of the centrifuge chamber (without a fluid transfer assembly) shown in FIG. 1C.

FIG. 2A is a top view of a bottom portion of an exemplary centrifuge chamber as described herein.

FIG. 2B is a side view of a bottom portion of an exemplary centrifuge chamber as described herein.

FIG. 2C is a top perspective view of a bottom portion of an exemplary centrifuge chamber as described herein.

FIG. 2D is a front view of a bottom portion of an exemplary centrifuge chamber as described herein.

FIG. 2E is a bottom view of a bottom portion of an exemplary centrifuge chamber as described herein.

FIG. 3A is a top view of a top portion of an exemplary centrifuge chamber as described herein.

FIG. 3B is a top perspective view of a top portion of an exemplary centrifuge chamber as described herein.

FIG. 3C is a bottom perspective view of a top portion of an exemplary centrifuge chamber as described herein.

FIG. 3D is a top view of the interior surface of the top chamber of an exemplary centrifuge chamber as described herein.

FIG. 3E is cutaway section B-B of the top portion of the exemplary centrifuge chamber shown in FIG. 3D.

FIG. 4A is a perspective view of an exemplary fluid transfer assembly as described herein.

FIG. 4B is an exploded view of the exemplary fluid transfer assembly shown in FIG. 4A.

FIG. 4C is a top view of the exemplary fluid transfer assembly shown in FIG. 4A.

FIG. 4D is the cutaway section A-A of the exemplary fluid transfer assembly shown in FIG. 4C.

FIG. 4E is detail B of the fluid transfer assembly shown in FIG. 4D.

FIG. 5A is a perspective view of an exemplary cradle as described herein.

FIG. 5B is an exploded view of an exemplary cradle as described herein.

FIG. 5C is a cutaway front view of an exemplary centrifuge chamber (with fluid transfer assembly) locked in position in an exemplary cradle as described herein.

### DETAILED DESCRIPTION

The embodiments disclosed herein relate to an improved centrifuge chamber, kits including a centrifuge chamber as described herein, and methods of separating desired particles from a suspension using a centrifuge chamber as described herein.

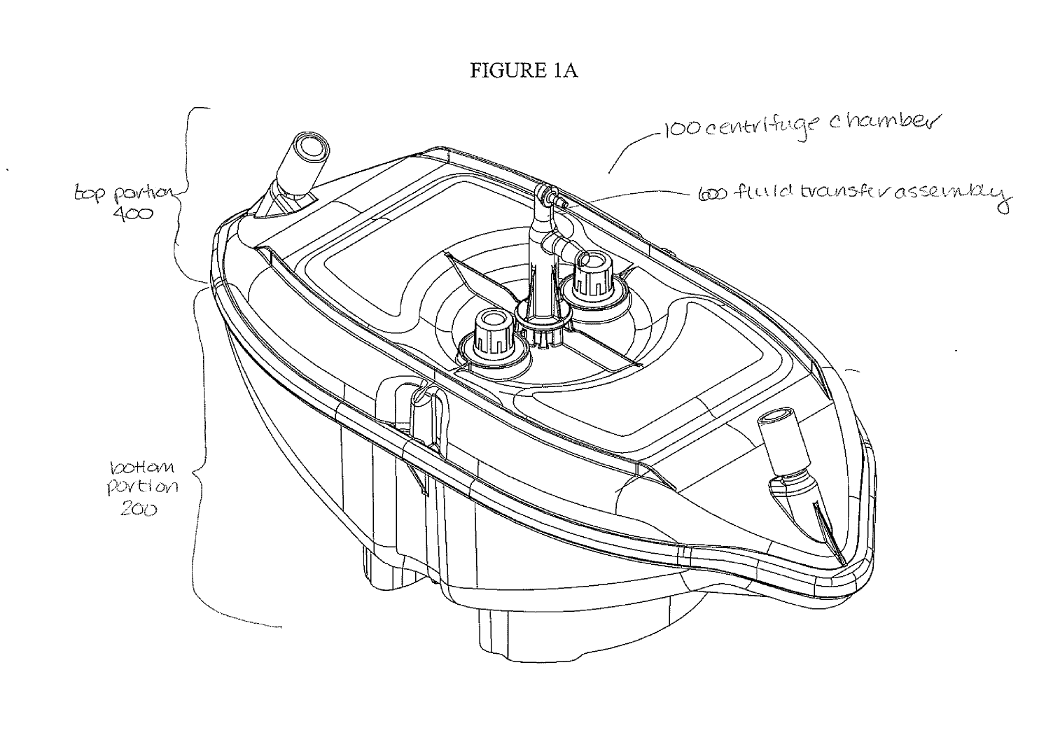

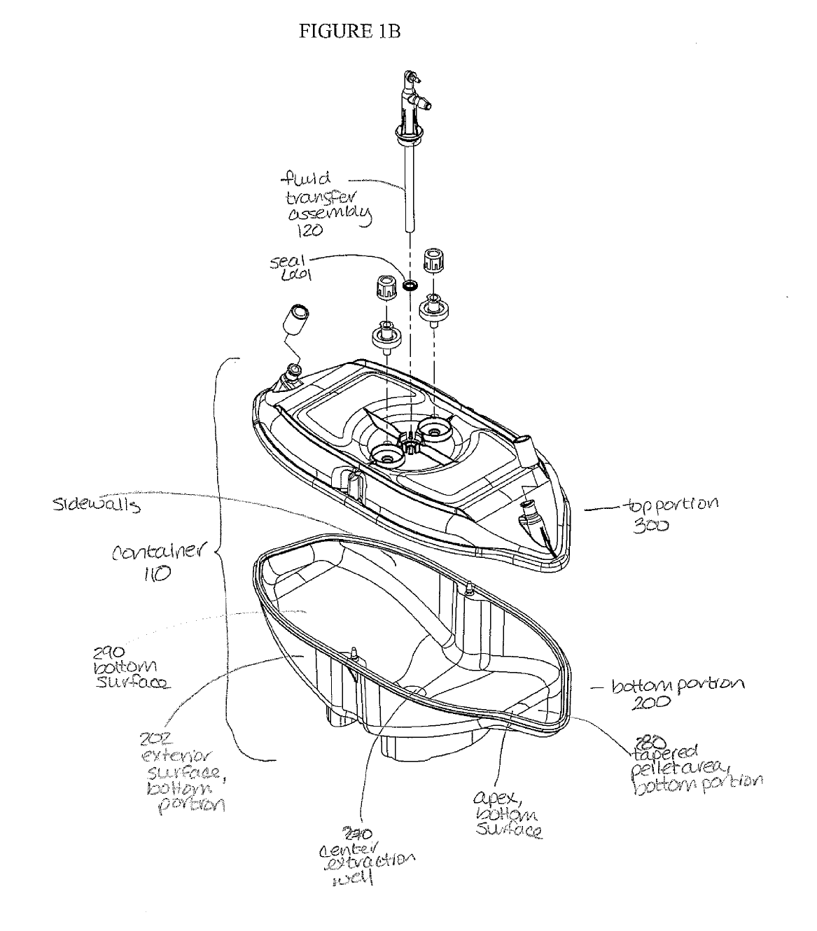

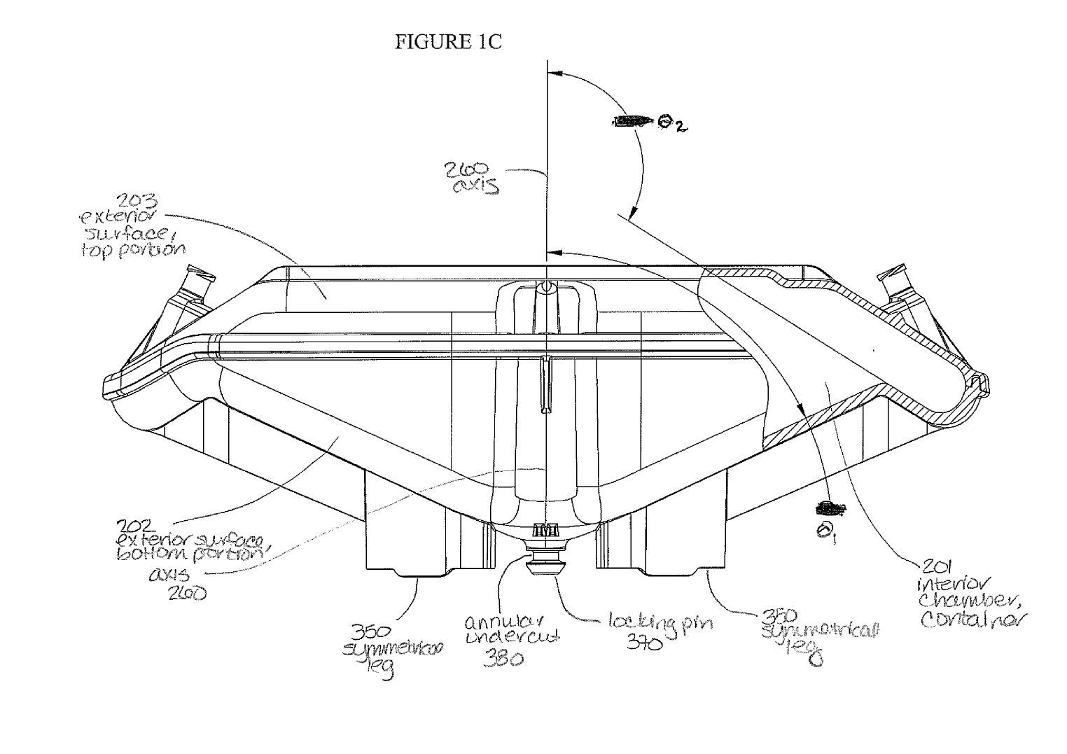

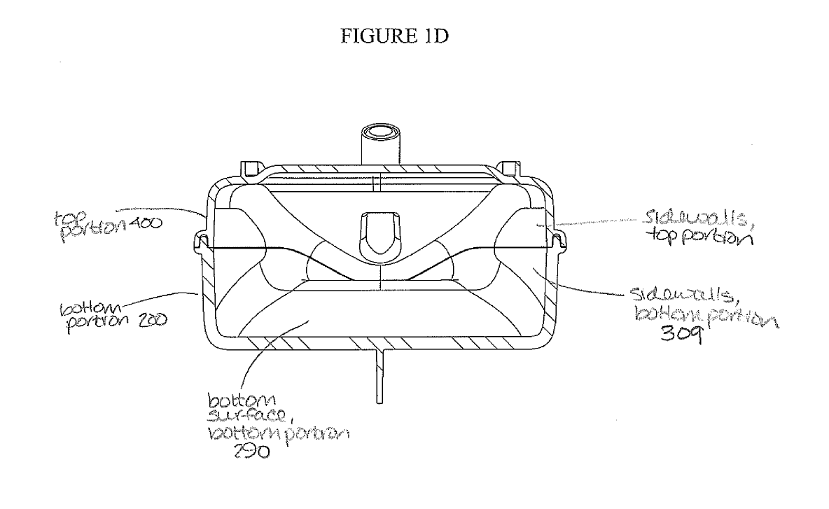

FIG. 1A depicts an exemplary centrifuge chamber 100 as provided herein. FIGS. 1A and 1B are perspective view and exploded views, respectively, of a centrifuge chamber 100 including a container 110, and a fluid transfer assembly 120 as described herein. The skilled person will readily appreciate that the container 110 shown in FIG. 1A can be unitary, e.g., made from a single mold and from a single material, or, alternatively, can be made from a top portion 300 and a bottom portion 200, e.g., as shown in FIG. 1B that are fixably attached using art-accepted means, e.g., with mattel pins, screws, via ultrasonic welding, glue, or the like, or any combination of art-accepted means.

The container can be made of different types of materials. Preferably, the container is manufactured using medical grade materials. Preferably, the container is manufactured of a hydrophobic material, so that particles in the suspension do not stick to or adhere to the inner surfaces of the inner chamber.

### Centrifuge Container

As shown in FIG. 1A, the containers described herein can include a top portion 300, and a bottom portion 200. Together, the top portion 400 and bottom portion 200 form a container having an interior chamber 101, and an exterior surface 102. Preferably, the surface of the interior chamber 101 is hydrophobic, to minimize interaction with biological material. In some embodiments, the surface of the interior chamber 101 comprises a hydrophobic coating, e.g., a wax, silicone, polyteraflouroethylene, or the like. In some embodiments, the surface of the interior chamber 101 can be coated with a substrate to allow for positive or negative selection of material (e.g., particles such as cells, extracellular matrix components, or the like), present in the suspension. By way of example only, the surface of the interior chamber 101 can be coated with nucleic acid binding agents, in order to separate nucleic acids present in a suspension from other particles in the suspension (e.g., cells, or the like). In alternative embodiments, the surface of the interior chamber 101 can be coated with cell-specific antibodies that recognize and bind antigens present for example, on desired cell populations in a cell suspension (e.g., tissue digested). These include both positive selection (selecting desired, target cells), negative selection (selective removal of unwanted cells from a cell suspension), or combinations thereof.

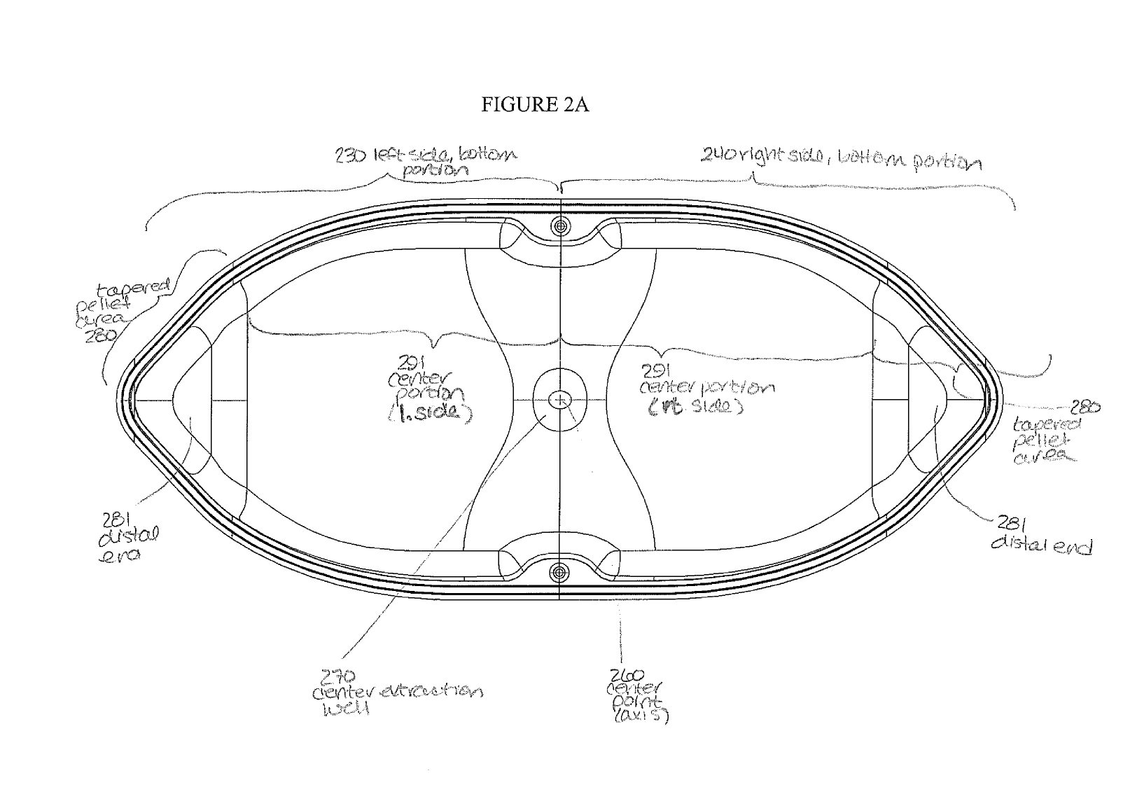

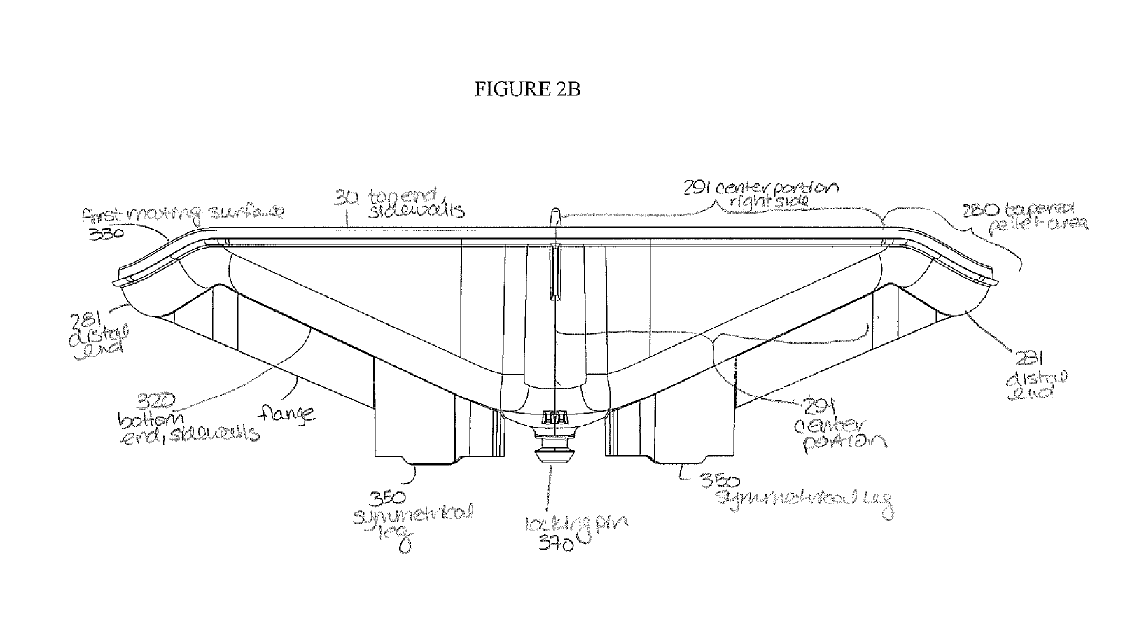

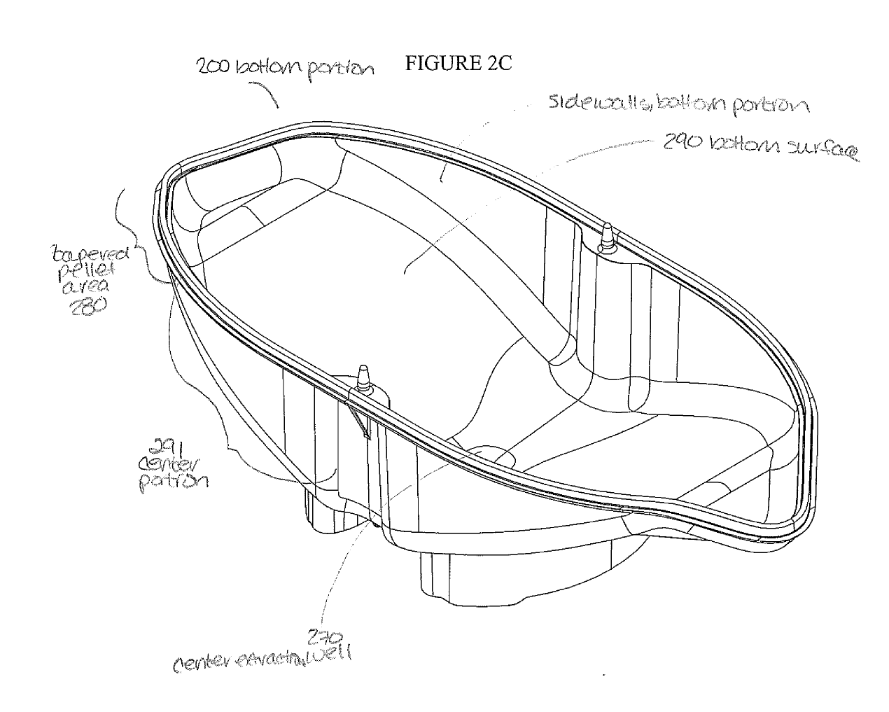

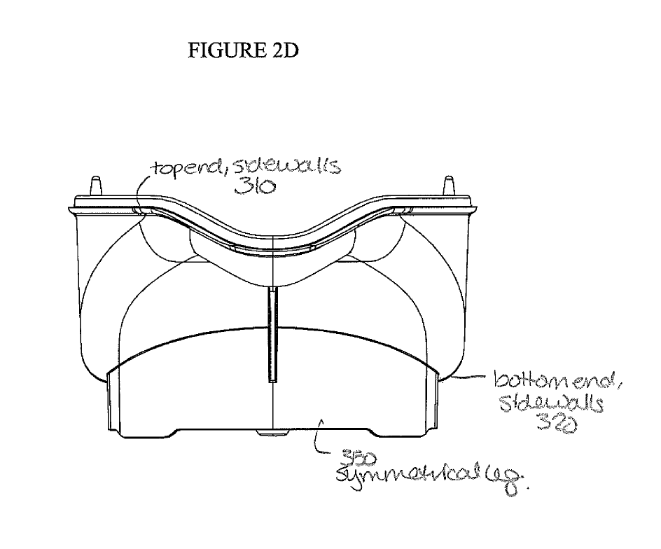

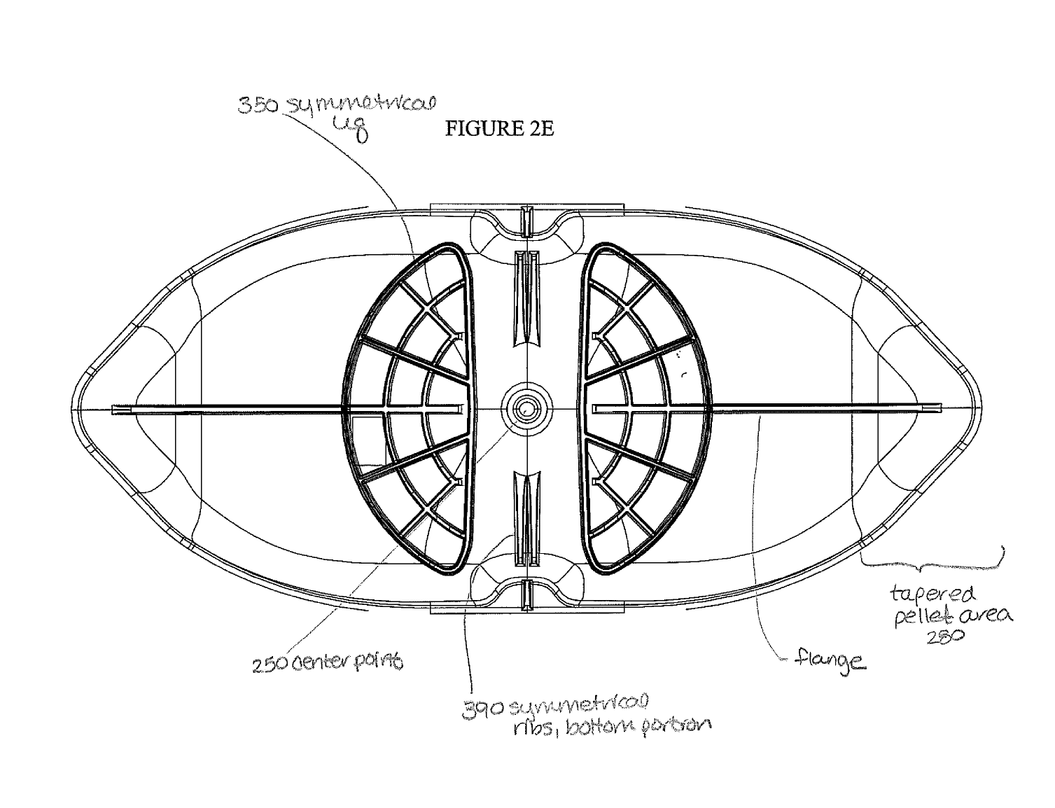

FIGS. 2A-2F depict an exemplary bottom portion 200 of a centrifuge container 110 as described herein. The bottom portion 200 of the centrifuge chamber 100 is bilaterally symmetrical, with symmetrical left and right sides. The bottom portion 200 has an interior chamber 201 and an exterior outer surface 202. Located in the center of the bottom portion 200 is a center point 250, which defines the axis 260 around which the container 110 rotates when in operation in a centrifuge as described herein. Surrounding the center point 250 in an annular fashion is a center extraction well 270 formed within the bottom surface 290 of the bottom portion 200. The center extraction well 270 is sloped inward and downward to the center point 250, and is configured to receive one end of a fluid transfer assembly 700 (optional). The bottom of the center extraction well 270 is the lowest point of the bottom surface 290. The center extraction well 270 can accommodate a volume of material within the interior chamber 101 of the container, whether or not a fluid transfer assembly 700 is present in the centrifuge chamber 100. Accordingly, the center extraction well 270 can accommodate a volume of a cell suspension, a resuspended cell population, or the like, as described in further detail below.

Each of the left 230 and right 240 sides of the bottom portion 200 is further defined by a center portion 291 that adjoins the left 230 and right sides 240, and distally located tapered pellet area ends 280, adjoining the center portions 291. Each center portion 291 is defined by a bottom surface and sidewalls 309. The bottom surface of each central portion is generally sloped at a first angle, θ1, that is less than 90° relative to the axis. The first angle Oladvantageously enables desired particles to travel up the sloped center portion and into the distal ends 281 of the container during centrifugation. Preferably, θ1 is between about 45° and 70°, relative to the axis 260, e.g., 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, or any number in between. The sloped bottom surface 290 may have a curve, i.e., it may have a convex shape, or it may have a straight or substantially straight slope towards the center point 250. Regardless, the apex 292 of the sloped bottom surface 290 is distal to the center point 250, where the center portion 291 adjoins the respective tapered pellet area end portion 280. The nadir of the sloped bottom surface 290 is the center point 250. In some embodiments, the sidewalls 309 of the center portions 291 are vertical or substantially vertical (i.e., parallel or substantially parallel to the axis 260).

The tapered pellet area end portions 280 of the bottom portion 200 are each formed from sidewalls 309 and a bottom surface 290. The bottom surface 290 of the tapered pellet area ends 280 are also generally sloped at a second angle, θ2, that is greater than 90° relative to the axis 260. The second angle advantageously enables the retention of particles resuspended in the distal ends 481 following centrifugation, as discussed in further detail below. θ2 also advantageously allows for retention of resuspension fluid (e.g., fluid from the initial suspension, or the like) in the tapered pellet area portions 280 following centrifugation, while pelleted material remains packed/pelleted in the distal ends 481 of the tapered pellet area portions. θ2 also advantageously allows for transfer of resuspended material (e.g., resuspended particles such as cells), into the center portion 291 of the interior chamber, e.g. by implementing a vortex motion around the axis 260, as discussed in further detail below. Preferably, θ2 is between about 105° and 140°, relative to the axis 260. For example, θ2 can be 105°, 106°, 107°, 108°, 109°, 110°, 111°, 112°, 113°, 114°, 115°, 116°, 117°, 118°, 119°, 120°, 121°, 122°, 123°, 124°, 125°, 126°, 127°, 128°, 129°, 130°, 131°, 132°, 133°, 134° 135°, 136°, 137°, 138°, 139°, 140°, or any number in between. The apex 292 of the sloped bottom surface 290 of the tapered pellet area end 280 is proximal to the center point 250, and defines the location where the tapered pellet area end portion 280 adjoins the center portion 291. As can be seen in FIG. 2A, the distal ends 281 of the tapered pellet area end portions 280 are generally U-shaped or V-shaped, although the skilled person will readily appreciate that the ends can be more or less rounded than the distal ends shown in the figures.

The sidewalls 309 (e.g., of the central portions and the tapered pellet area end portions) have a top 310 and a bottom end 320, wherein the bottom end 320 of the sidewalls 309 adjoin the bottom surface 290 of the bottom portion 200, and wherein the top end 310 of the sidewalls 309 form a first mating surface 330. The first mating surface 330 formed by the top end 310 of the sidewalls has the same shape and has complementary angles and slopes to a second mating surface 340 defined by the sidewalls of the top portion 400, as discussed below. The top end 310 of the sidewalls of the center portion 291 can be perpendicular to the axis 260, while the top end 310 of the sidewalls of the tapered pellet area end portions 280 can be angled, e.g., sloped at the angle θ2 relative to the axis 260. The top portion 400 and bottom portion 200 of the container 110 can be fixably attached by coupling the first 330 and second 340 mating surfaces.

In some embodiments, the bottom portion 200 forms part of a locking means that secures the container into a cradle that forms part of a centrifuge. For example, in some embodiments, the exterior surface 202 of the bottom portion 200 can include two or more symmetrical legs 350, as depicted in FIG. 2F. As depicted in the embodiment shown in FIG. 2F, the legs 350 are each equidistant from each other and are equidistant to the center point 250 of the bottom portion. In some embodiments, the legs 350 can have a patterned surface, e.g., as shown in FIG. 2F. The patterned surface can be formed of ridges. The patterned surface can be complementary to a patterned surface in a cradle. Full or partial complementarity between the legs 350 of the exterior surface 202 and a patterned surface in a cradle can advantageously function to ensure proper loading of the container 110 in the cradle of the centrifuge, and/or secure the container 110 in the cradle of the centrifuge. In some embodiments, the ridges can be load-bearing. That is, the ridges can be configured to withstand rotational forces, e.g., exerted from a complementary structure of the cradle upon which the container sits, when the centrifuge rotates the chamber 100 about the axis 250 (e.g., when the cradle is rotated up to 500; 1,000; 2,000; 2,500; 3,000; 5,000; up to 10,000, up to 15,000, up to 20,000, or greater rpm's, e.g., such that the suspension is subjected to between 100-1,000 g's at the distal ends of the chamber, and preferably between 400-600 g's at the distal ends of the chamber).

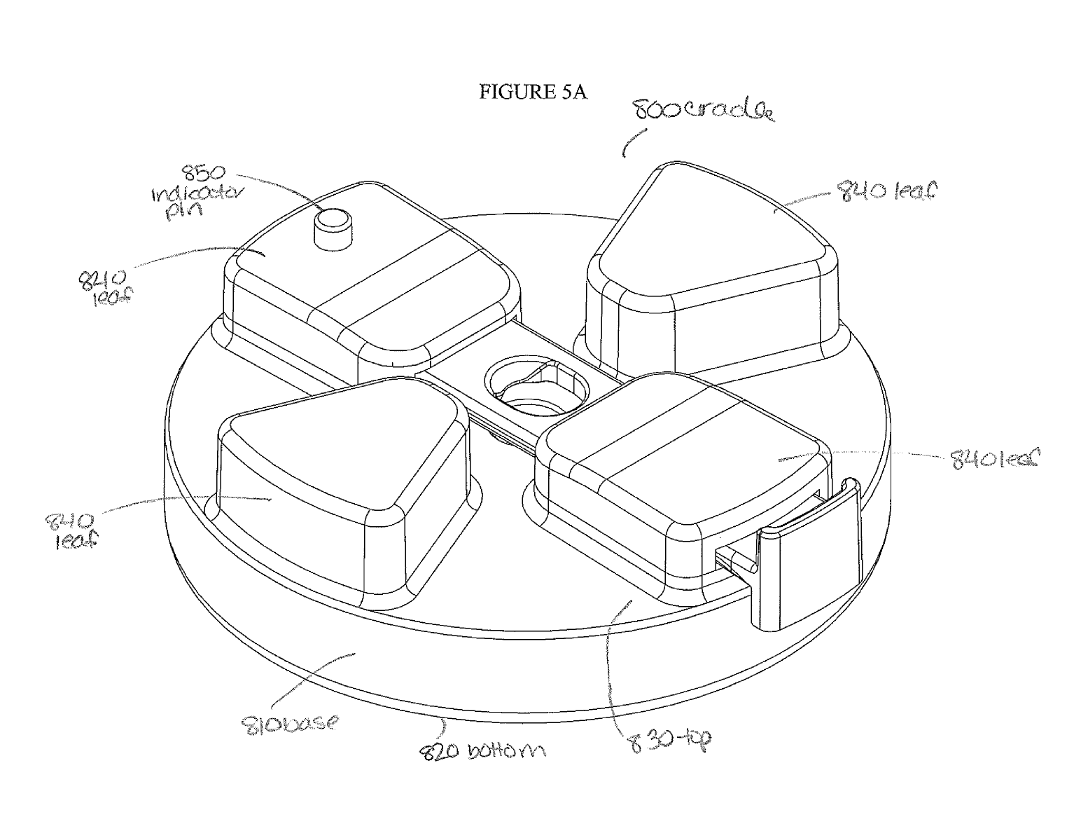

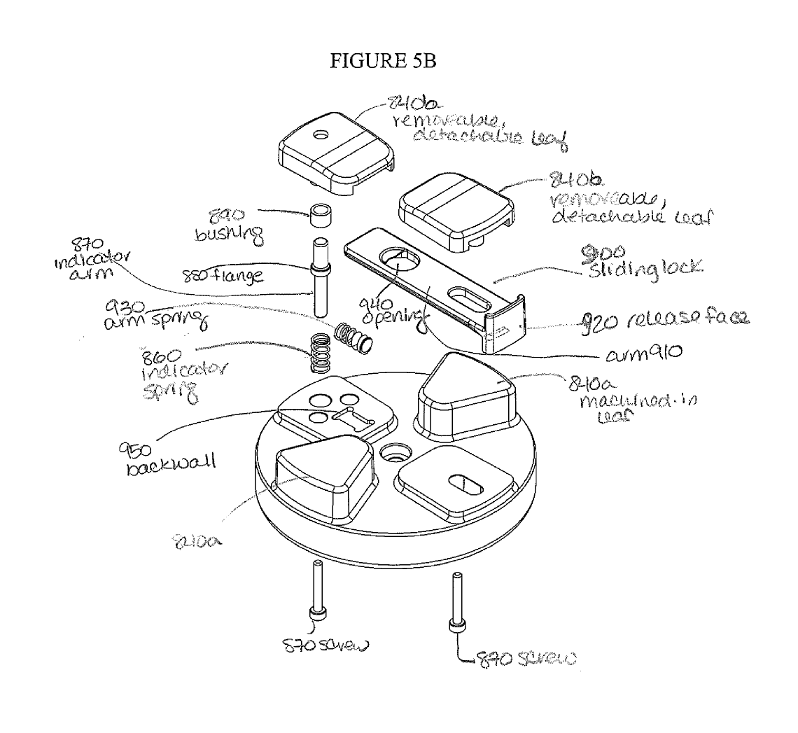

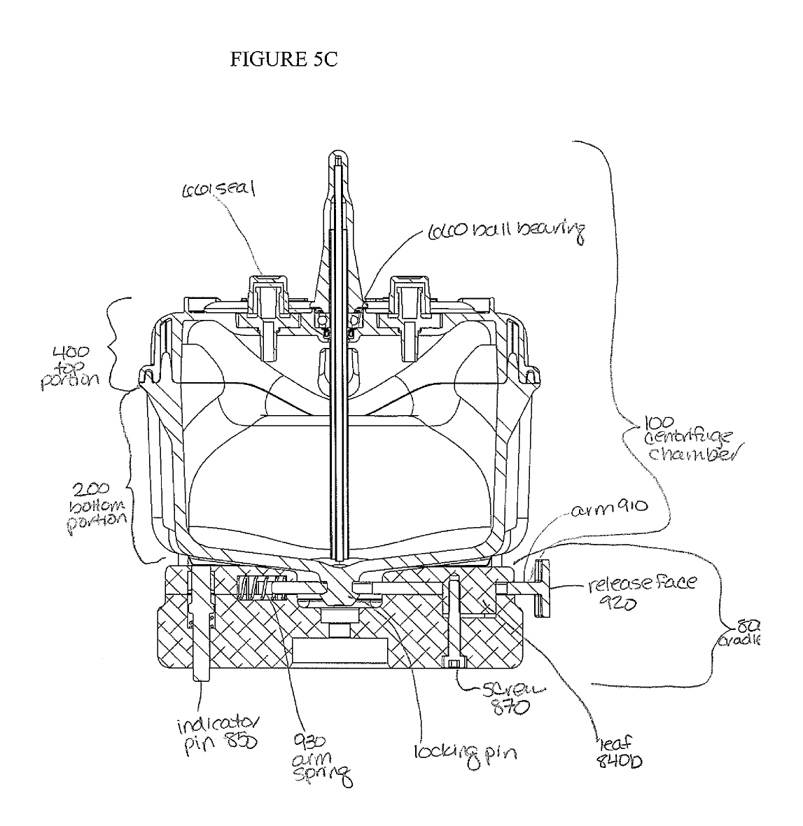

An exemplary cradle 800 useful in the embodiments disclosed herein is depicted in FIG. 5A. The cradle 800 can include a base 810 having a bottom 820 and a top 830 with one or more (e.g., 1, 2, 3, 4, 5, 6, 7, 8, 9, 10) leaves 840 extending from the top of the base, and which can each be complementary or accommodate the shape of one or more respective legs 350 of the container 110. As shown in FIG. 5A, in embodiments where there are multiple leaves 840, the leaves do not have to be the same shape. In some embodiments, one or more leaves 840a can be machined into the base 810 of the cradle. In some embodiments, one or more leaves 840b can include a detachable portion that is fixably attached to the base using any art accepted means. By way of example only, detachable leaves 840b shown in FIG. 5B are fixably attached to the cradle base 810 via screws 870.

The exemplary cradle 800 shown in FIG. 5A includes an indicator pin 850. By way of illustration, the embodiments shown in FIGS. 5A-5C illustrate the indicator pin 850 positioned in one of the leaves 840 of the cradle 800, although the skilled person will readily appreciate that the location of the indicator pin can be elsewhere in the cradle. The indicator pin 850 shown in FIG. 5B comprises a spring-loaded structure that includes an indicator spring 860, and indicator arm 870 with a flange 880, and optionally a bushing 890, although the skilled person will readily recognize that numerous different configurations of an indicator pin can be used in the embodiments disclosed herein. In the embodiment shown in FIGS. 5A-5C, symmetrical ribs 390 on the exterior surface of the bottom portion 202 displace the indicator pin 850 by pushing on spring 860 when the container is positioned within the cradle 800.

In some embodiments, the exterior surface 202 of the bottom portion 200 can include one or more symmetrical locking pins 370, e.g., as depicted in FIG. 2B, that can function to secure the container 110 into a centrifuge cradle, e.g., that can function as part of a locking means. In the embodiment shown in FIG. 1C, the locking pin 370 can include an annular undercut 380, complementary to a spring-loaded structure in the cradle, as discussed in further detail below. The skilled person will recognize that many different configurations of a locking means to lock the centrifuge chamber/container into a cradle can be used in the embodiments disclosed herein, and that the legs 350 and the pin 370 are exemplary only. For example, the locking means can comprise a snap, an automated constraining lock, an electrical lock, a pneumatic constraining lock, or the like.

An embodiment showing a locking mechanism that comprises a locking pin 370 coupled with a cradle 800 is depicted in FIG. 5C. In the illustrative, exemplary embodiment, the cradle 800 can include a sliding lock 900 that has an arm 910 having a release face 920 on a first end of the arm, and coupled to a front end an arm spring 930 on the second end of the arm. The back end of the arm spring 930 rests against a backwall 950 within the cradle, such that the arm spring 930 becomes compressed against the backwall 950. In the embodiments shown in FIG. 5C, the arm comprises one or more openings 940 that accommodates and holds into place the leg of the locking pin 370 via movement of the arm 910 mediated by decompression of the arm spring 930.

In some embodiments, the exterior surface 202 of the bottom portion 200 can include an indicator means, which functions to generate detectable signal when the centrifuge chamber is properly loaded and locked in a centrifuge chamber. In some embodiments, the indicator means can be one or more symmetrical ribs 390 located on the exterior surface 202 of the bottom portion 200. For example, when the centrifuge chamber 100 is positioned correctly and locked into the centrifuge cradle, the one or more symmetrical ribs 390 can push on an indicator pin 850 within the cradle, causing the pin to change position and actuate an inductive sensor, as discussed in further detail below. The change in position can be detected, e.g., by an electrical, optical, or other means, thereby indicating that the centrifuge chamber 100 is properly loaded and locked within the cradle.

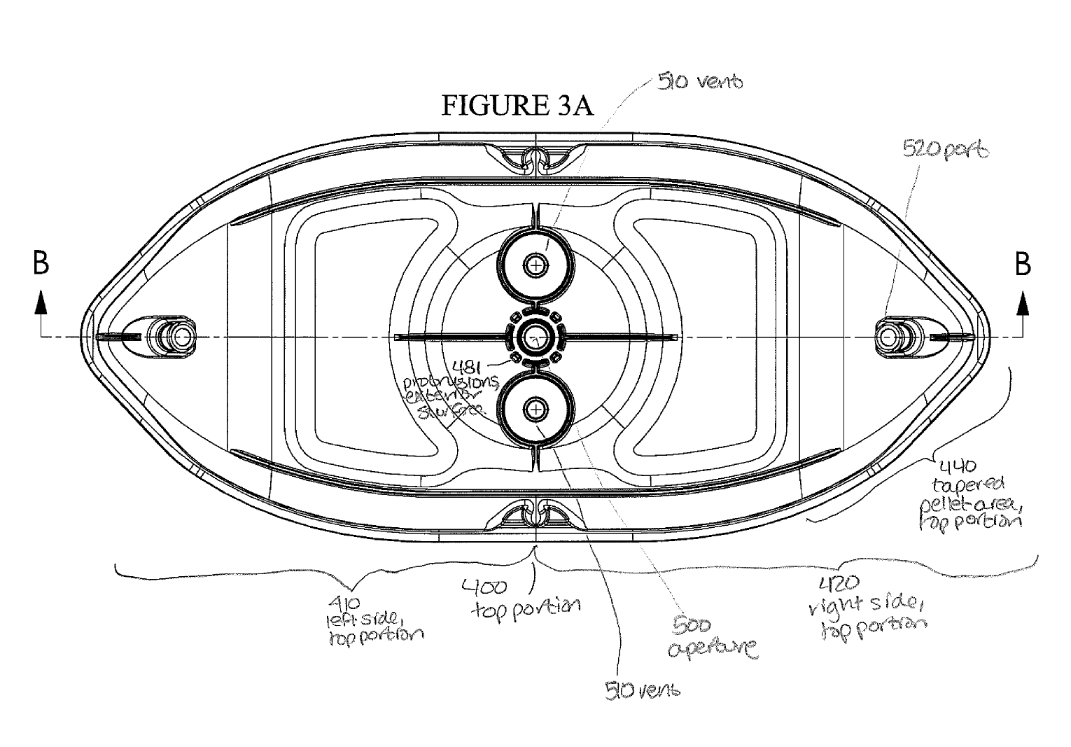

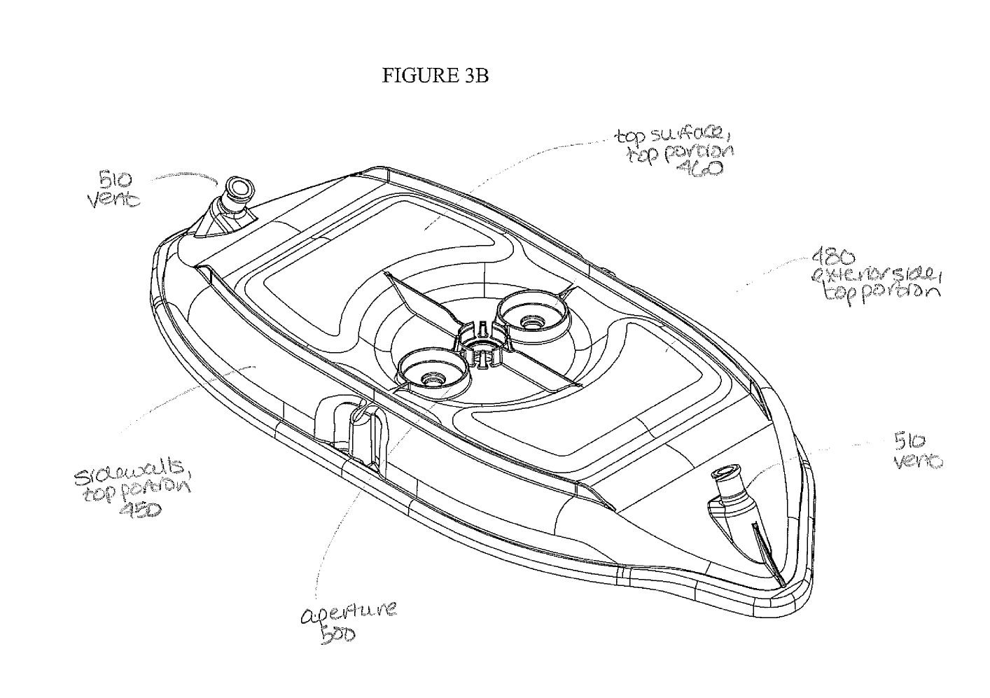

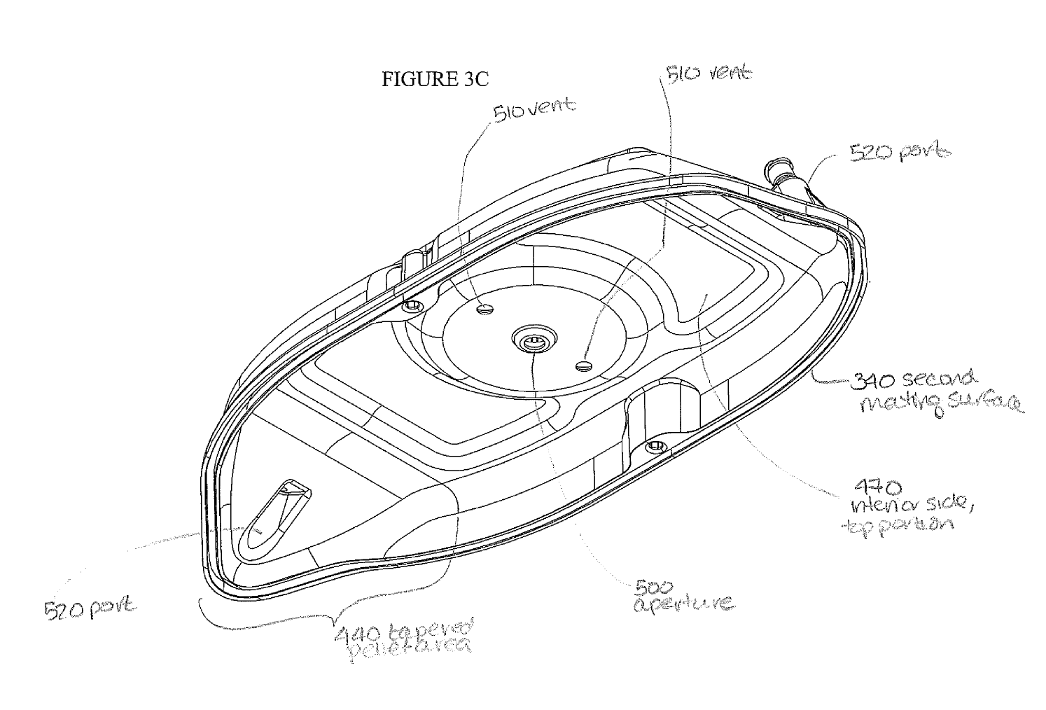

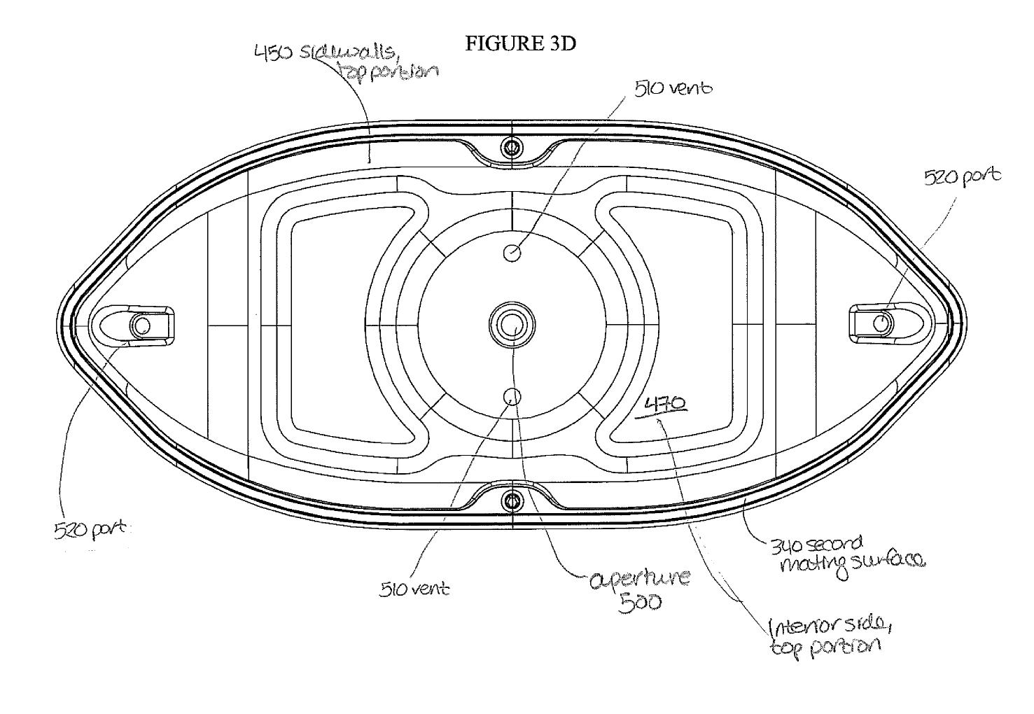

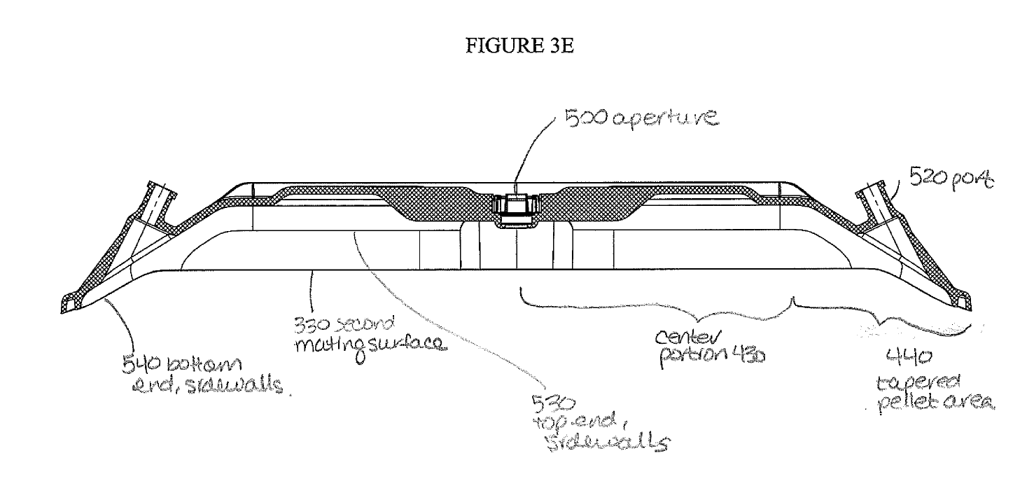

Various views an exemplary top portion 400 of a container 110 as described herein are shown in FIGS. 3A-3F. The top portion 400 is bilaterally symmetrical, with left and right sides. As with the bottom portion 200, each of the left 410 and right sides 420 of the top portion has a center portion 430 adjoined to a tapered pellet area end portion 440. The top portion 400 has sidewalls 450 and a top surface 460 having an interior side 470 that forms the top of the interior chamber, and exterior side 480, that forms part of the exterior surface 102 of the chamber 100. The center of the top portion defines a center point 490, e.g., defining the axis 260 around which the centrifuge chamber 100 rotates. In some embodiments, the top portion 400 has an aperture 500 around the center point 490, e.g., which can accommodate structures such as a straw of a fluid transfer assembly 700, as discussed in further detail below.

The sidewalls 450 of the top portion 400 have a top end 530 and a bottom end 540. The top end 530 adjoins the top surface 460 of the top portion 400, and the bottom end 540 forms the second mating surface 340 discussed above. The second mating surface 340 has the same shape as, and complementary angles to, the first mating surface 330 of the bottom portion 200. The first 330 and second mating surfaces 340 can be joined together using any art-accepted means (e.g., glue, screws, clips, nails, bonds, or the like, including combinations thereof), thereby forming the container 110.

In some embodiments, the top portion 400 can optionally include one or more vents 510, e.g., sterile vents, coaxial and symmetrically positioned around the axis 260, e.g., as depicted in FIG. 3D. The vents (e.g., sterile vents 510) can advantageously function to allow for the sterile displacement of air, e.g., when material is transferred into or out of the interior chamber 101 of the container 110. In some embodiments, the vents can comprise a filter to maintain sterility of the interior chamber 101. For example, in some embodiments, the vents 510 can comprise filters less than or equal to 5 μm.

In some embodiments, the top portion 400 also optionally includes one or more ports 520 that allow sterile access into and out of the interior chamber 101, thereby providing for sterile ingress and egress of material into and out of the interior chamber 101. By way of example only, each of the left 410 and right 420 sides of the top portion 400 can include symmetrical ports 520. Exemplary ports 520 located in the tapered pellet area ends 440 of the right and left sides of the top portion 400 are shown in the top portion depicted FIG. 3D. The exemplary ports 520 shown in FIG. 3D can enable sterile access, e.g., via a syringe or other means, to the distal portion of the tapered pellet area end 280/440 of interior chamber 101 of the container 110. As such, the ports 520 can allow for resuspension and/or removal of material (e.g., desired cell populations or the like), pelleted at the distal ends of pellet area ends of the container 110. The ports 520 can also allow for addition of material (e.g., a resuspension buffer or the like), if desired, into the tapered pellet area end portion 280/440 of the interior chamber 101. The exemplary ports 520 depicted in FIGS. 3A-3E are angled upwards and towards the axis 260 in the center point. The ports 520 can be configured to accept a locking luer, or the like, that allows for sterile connection of an off-the-shelf syringe or other apparatus for transfer or collection of material into and out of the interior chamber 101. In some embodiments, the ports 520 can comprise a filter. For example, in some embodiments, the ports 520 can comprise a filter that retains cell aggregates and passes single cells—e.g., a macrosyringe filter having a mesh size of 20-60 μm, e.g., 30, 35, 40, 45, 50, 55, 60 μm, or greater, or any value in between. Accordingly, in embodiments wherein resuspended material is removed from the tapered pellet area end portions 280 via ports 520, single cell suspensions can be removed via the port, while any aggregates remain on the interior chamber side of the port 520.

As discussed above, the bottom surface 290 and optionally the top surface 460, of the tapered pellet area ends 280/440, respectively, of the container 110 slope downwards and away from the axis 260, e.g., at the angle θ2. The angle of the tapered pellet area ends 280/440 of the interior chamber 110 advantageously enable the retention of unpelleted material within the tapered pellet area ends. Due to the slope of the tapered pellet area end, gravitational forces provide for the retention of a volume of unpelleted material within the tapered pellet area end portions of the interior chamber 101 following centrifugation. As explained in further detail below, the unpelleted material within the tapered pellet area end portions can advantageously be used to resuspend material pelleted in the distal ends, e.g., by rotating or shaking the chamber (vortexing mode) about the axis, as discussed in further detail below.

The contour of the distal ends of the tapered portions of the interior chamber can be substantially U-shaped or V-shaped, e.g., as shown in FIG. 2A. The substantially U-shaped or V-shaped contour is designed to advantageously provide for resuspension of pelleted material therein, as well as removal of resuspended material within the tapered pellet area ends, into the central portion (e.g., the center extraction well 270) of the interior chamber 101, as discussed in further detail below. In some embodiments, resuspension of pelleted material is achieved by rotating the chamber 100 about the axis 260 in a vortex mode, such that the pelleted material is resuspended in the residual fluid located within the tapered pellet area ends following centrifugation. In some embodiments, the resuspended material is transferred to the center extraction well 270 in the bottom surface of the container by rotating the chamber about the axis 260 in a transfer mode, e.g., by changing the direction of and/or stopping the rotation of the chamber about the axis 260. The transfer mode causes resuspended material within the tapered pellet area end portions of the interior chamber 101 to travel up the bottom surface 290 of the tapered pellet area end potion 280, and down into to the center extraction well 270 of the center portion of the interior chamber 101.

### Fluid Transfer Assembly

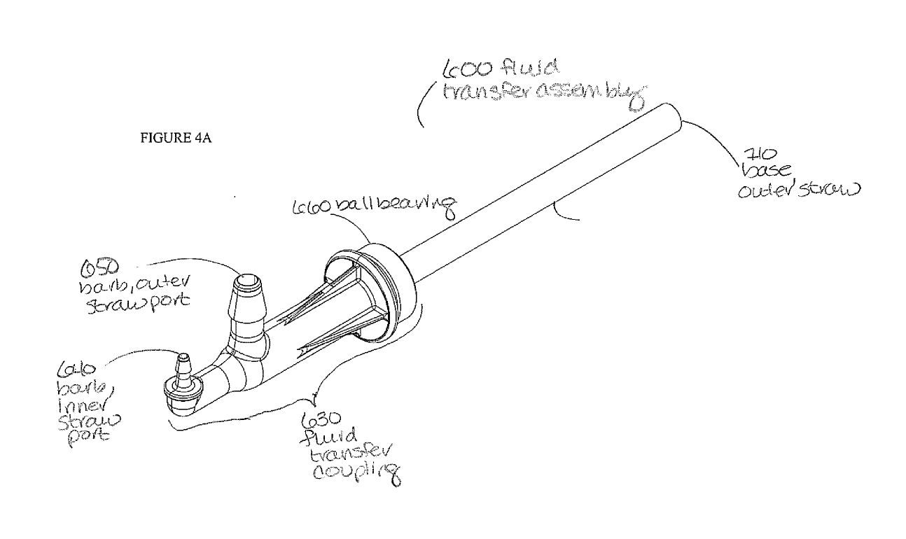

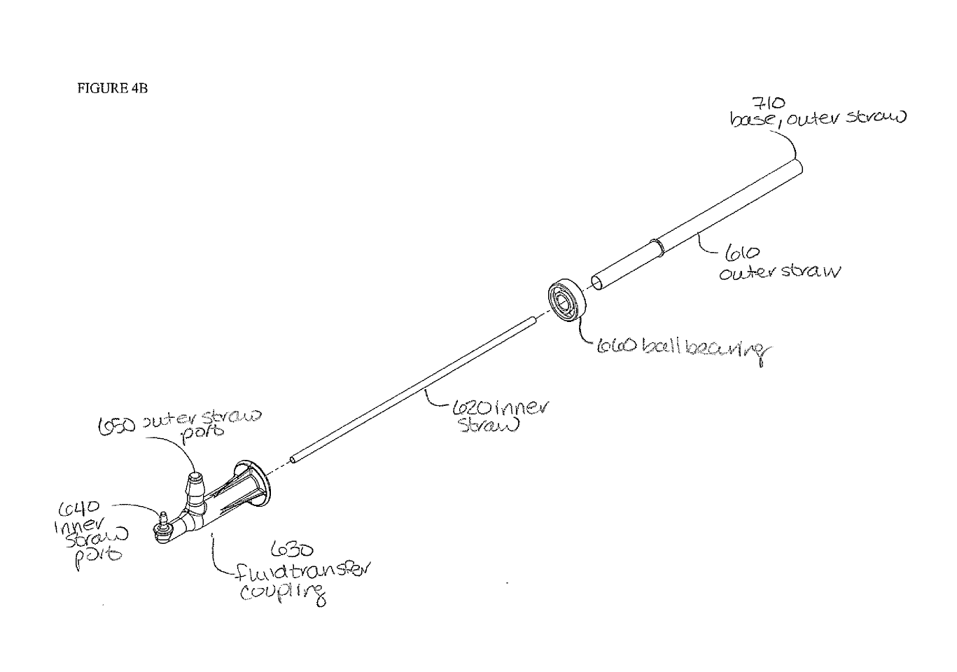

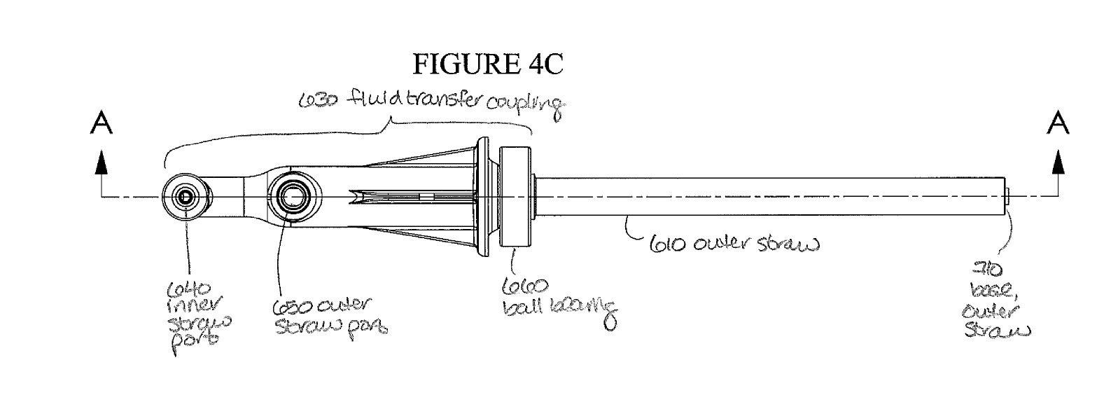

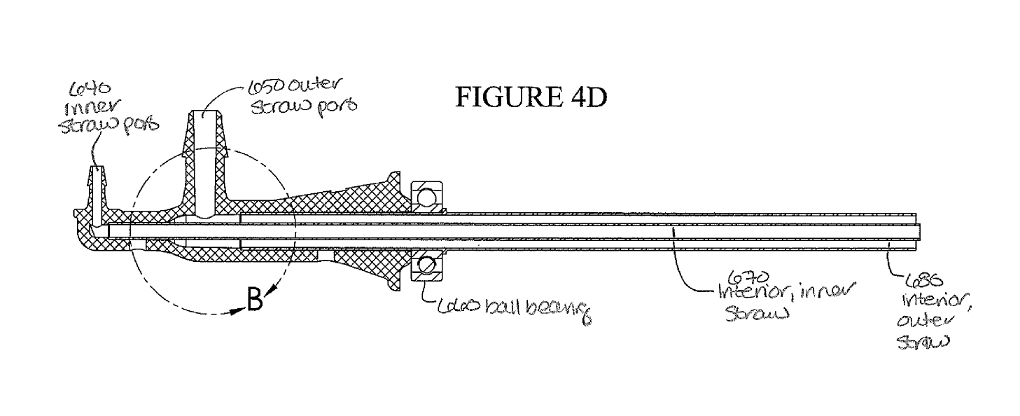

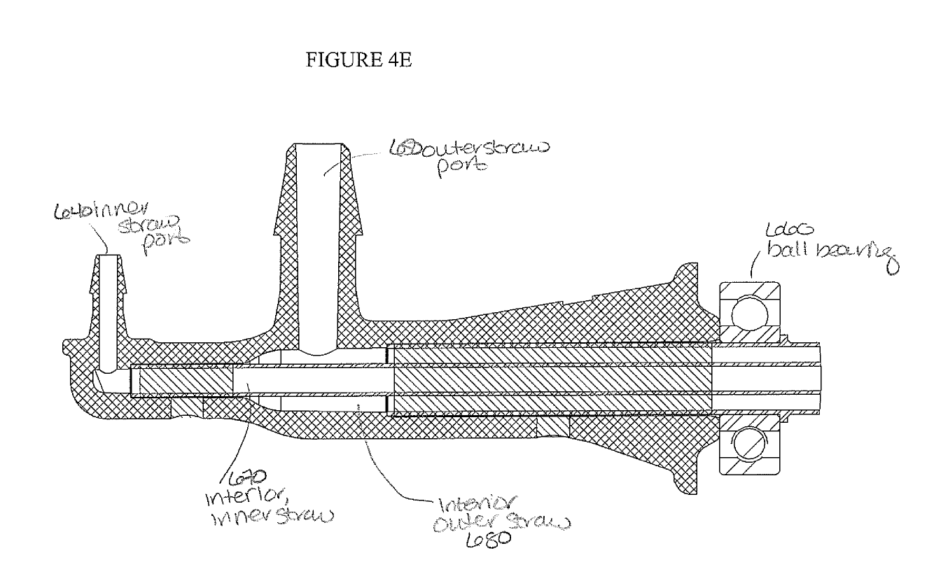

The centrifuge chambers provided herein can also optionally include a fluid transfer assembly, configured to permit ingress and egress of material in and out of the interior chamber of the container. An exemplary fluid transfer assembly 600 is shown in FIGS. 4A-4E. The fluid transfer assembly 600 can include a substantially cylindrically shaped outer straw 610 having an interior 680 and an exterior 690, and a coaxially located, substantially cylindrically shaped inner straw 620 having an interior 670 and an exterior 700 housed within the outer straw 610. Each of the inner straws 620 and outer straws 610 has a head and a base. The base ends 710, 720 are received in the center extraction well 270 of the bottom portion 200 of the container 110 when the fluid transfer assembly 600 is positioned within the chamber 100. The assembly 600 can include a fluid transfer coupling 630 located at the head of the inner 730 and outer 740 straws, that joins the interior of the outer straw 680 and the interior of the inner straw 670, respectively, to conduits that provide a sterile, closed pathway to chambers capable of housing material to be directed into or out of the interior chamber of the container. As shown in FIG. 4A, the fluid transfer coupling 630 can include a first 640 and second barb 650 that form first and second ports, respectively, that are fluidically coupled to the interior of the inner straw 670 and the interior of the outer straw 680, respectively. When positioned within the centrifuge chamber 100, the first 640 and second 650 ports provide for access of material within the center extraction well 270 of the bottom portion 200 via the interior of the inner 670 and outer 680 straws on their base ends (720, 710), and to the first 640 and second 650 ports on the head end (740, 730). When positioned within the container 110, the base ends of the inner 720 and outer 710 straws is located within the center extraction well 270 in the bottom surface of the bottom portion 200, and the inner and outer straws are annularly positioned with respect to the axis 260. As such, material (e.g., fluids, cell suspensions, a desired cell population or the like), can be transferred into or out of the center extraction well of the interior chamber 101 via the first 640 and second 650 ports. In some embodiments, the second port 650 that provides access to the inner chamber via the inner straw 620 can comprise a filter. For example, in some embodiments, the ports 520 can comprise a filter that retains cell aggregates and passes single cells—e.g., a filter having a mesh size of 0.2-0.5 μM. Accordingly, in embodiments wherein resuspended material is removed from the center extraction well 270, single cell suspensions can be removed via the second port 650, while any aggregates remain do not pass through the second port 650. can comprise a filter. For example, in some embodiments, the port 650 can comprise a filter that retains cell aggregates and passes single cells—e.g., a filter having a mesh size of 10-70 μM, e.g., 10, 15, 20, 25, 30, 35, 40, 45, 50, 55, 60, 65, 70 μm, or greater, or any value in between. Accordingly, in embodiments wherein resuspended material is removed from the center extraction well 270 via the second port 650 of the inner straw 620, single cell suspensions can be removed via the port, while any aggregates do not pass through port 650. In a similar manner, the first port 640 that provides for the ingress and egress of material through the outer straw 610 can include a filter. The inner 720 and outer 710 straws can be fixably attached to the fluid transfer coupling 630 using any art-accepted means, e.g., glue, soldering, press fit, or the like.

The fluid transfer assembly can include a ball bearing 660 that fits around the exterior surface of the outer straw 690 between the fluid transfer coupling 630 and the center point aperture 500 of the top surface 460 of the top portion 400 of the container. The ball bearing 660 is configured to allow for rotation of the container 110 around the fluid transfer assembly 600 and coaxial axis 260, while the outer and inner straws (610, 620), as well as the fluid transfer coupling 630 remain stationary. The exemplary ball bearing 660 depicted in FIG. 4B is ring-shaped ball-bearing. In some embodiments, the fluid transfer assembly can include a seal 661 that is ring-shaped and that fits underneath the ball bearing of the fluid transfer coupling, and which forms an air-tight seal with the container.

In some embodiments, the exterior surface 480 of the top portion 400 has radially symmetrical protrusions 481 around the center point aperture 500, e.g., as depicted in FIG. 3D. In some embodiments, the protrusions 481 can function as a locking means that secures the fluid transfer assembly 600 in position within the container 110. In some embodiments, the ball bearing 660 is snapped into place in the protrusions 481. In some embodiments, a portion of the ball bearing 660 fits within an undercut of the fluid transfer assembly, and a portion of the seal fits within the protrusions 481 on the exterior surface 480 of the top portion 400. Seal 661 fits underneath the ball bearing 660 to seal the interior contents of the chamber 100.

Methods of Isolating Desired Particulates from Suspensions

Provided herein are methods of isolating desired particles from suspensions, using the centrifuge chamber described herein. For example, some embodiments provide for the isolation of a desired cell population from a cell suspension, e.g., the isolation of stromal vascular cells from disaggregated adipose tissue, stromal cells from bone marrow, and the like.

To separate desired particles from a suspension, a centrifuge that has a cradle with which the centrifuge chamber described elsewhere herein mates, and which also includes a driver that provides rotational force around the axis of the container, is provided. The centrifuge chamber can be inserted and locked into position in the cradle. For example, insertion and locking of the centrifuge chamber into the centrifuge can be accomplished by mating a patterned surface located on the exterior surface 202 of the bottom portion 200 of the container 110 (e.g., on at least two symmetrical legs 350 as described above), with a complementary patterned surface present on a cradle for the centrifuge chamber 100. In some embodiments, the patterned surface of the symmetrical legs 350 can function to strengthen the container 110. Alternatively, or in addition to, the patterned surface (e.g., on legs extending from the exterior surface of the bottom portion 200), the insertion and locking in position of the centrifuge chamber 100 into a cradle can include locking a pin 370, e.g., that extends from the center point 250 of the bottom portion 200, described above, into a complementary structure (e.g., such as a spring-loaded structure) in the cradle. As discussed above, in some embodiments the cradle of the centrifuge and the container 110 together provide an indicator means (e.g., by movement of a pin or the like in the cradle as described above). In such embodiments, the methods can include the step of receiving an indication that the container 110 is correctly positioned and locked within the cradle.

In some embodiments, the bottom portion 200 can optionally include flanges 361 that extend from each symmetrical leg 350 to the exterior surface of the tapered pellet area end 280, e.g., as depicted in FIG. 2C. In some embodiments, the flanges 361 provide structural support for the container 110.

The suspension can be transferred into the interior chamber 101 of the container 110. In some embodiments, transfer of the suspension to the interior chamber 101 of the container can be achieved using one or both ports 520 in the tapered pellet area ends 440 of the top portion 400 of the container 110, if present. Alternatively, or in addition to, use of ports 520 in the tapered pellet area ends 440, material can be transferred to the interior chamber 101 of the container 110 via a fluid transfer assembly 600, e.g., via a outer straw port 650 that provides access to the interior portion of the outer straw 680 of a fluid transfer assembly 600 as described herein. As such, ports 520 located on the tapered pellet area ends 440 of the top portion, or a fluid transfer assembly 400 as described herein, can be used to introduce material (e.g., cell suspensions or the like), into the interior chamber 101 of the container 110.

Once the fluid suspension is transferred into the interior chamber 101 of the container, a separation step is then performed, by causing the container 110 to rotate around the axis 260 (e.g., via any art-accepted drive in the centrifuge). The rotation generates a centrifugal force within the chamber 110 causing particles to move radially outward from the center point 250 in a density-dependent manner. The speed at which the container is rotated about the axis 260 for separation of desired materials is readily apparent to the skilled person. By way of example only, stromal vascular cells can separated from undesired material by centrifugation at 1000g. Due to the sloped configuration of the bottom surface 290 of the center portion 291 of the interior chamber 101, particles (e.g., desired cell populations or the like) will be forced against and up the slope to the apex, and will then migrate downwards to the distal ends 281 of the tapered portions 280 of the chamber, where the particles (e.g., desired cell populations or the like) pellet. As discussed above, due to the downward and outward—facing slope of tapered pellet area ends, gravitational forces cause a portion of fluid to be retained within the tapered pellet area ends along with the pelleted material following centrifugation. The less dense portion of the suspension will substantially remain in the center portion 291 of the interior chamber 101. As such, the desired particles are separated from less-dense portions of the suspension. Thereby, the method provides for the separation of a population of cells from undesired components within a cell suspension.

In some embodiments, the methods include the step of removing the desired particles pelleted in the distal, tapered pellet area ends 281 of the container. In embodiments wherein the centrifuge chamber 100 includes ports 520 providing sterile access to the tapered pellet area ends 440 280 of the top portion, a capture device (e.g., a syringe, or the like) connected to the port 520 can be used to introduce material into and/or remove material out of the tapered pellet area end of the interior chamber. Accordingly, some embodiments include the step of introducing a resuspension material into one or both of the tapered pellet area ends of the interior chamber, and resuspending pelleted material located in the distal ends 281 of the tapered portions with the resuspension material. In some embodiments, the pelleted material is merely resuspended in the residual fluid retained within the tapered portion 280/440 following centrifugation. In some embodiments, the removing step involves collecting resuspended material through one or both of the ports 520 located in the top portion 400 of the chamber 100.

In some embodiments, the methods include the step of resuspending material pelleted in the tapered pellet area ends, and transferring the resuspended material to the center extraction well 270 in the center of the bottom portion 200, where it is subsequently removed. In these embodiments, the methods include the step of removing undesired material in the suspension, e.g., undesired components that have been separated from the desired particles (e.g., desired cell populations, or the like), from the center portion 291 of the interior chamber 101, prior to transferring the resuspended material (comprising the desired particles) to the center portion 291of the interior chamber 101. Accordingly, some embodiments the methods provide a step of removing undesired components and/or material via an outer straw 610 in a fluid transfer apparatus 600 as described herein. Removal of undesired components through the outer straw port 650 connecting to the interior 680 of the cylindrically shaped outer straw of the fluid transfer system can be accomplished by virtue of applying a vacuum (manual or automatic) to draw fluid from the center extraction well 270 up into and through the interior of the outer straw, through the outer straw port 650 in the fluid transfer coupling 630.

Once undesired material is removed from the center portion 291 of the interior chamber 101, the pelleted material present in the tapered pellet area end portions 280/440 of the container 110 can be resuspended. As discussed above, resuspension of the pelleted material can be resuspended in fluid introduced into the tapered pellet area ends 280/440 through ports 520 located in the tapered pellet area ends 440 of the top portions 400 following centrifugation, or the pelleted material can be resuspended in residual fluid present in the tapered pellet area ends 280/440 following centrifugation. Regardless, resuspension of the pelleted material can be manual or automated. For example, in some embodiments, a syringe coupled to a port 520 in the tapered pellet area end of the top portion 400 can be used to manually resuspend pelleted material. Alternatively, material can be resuspended in an automated fashion via a syringe or other means coupled to a port in the tapered pellet area end 440 of the top portion 400. In yet other embodiments, the methods include the step of automatically resuspending material in the tapered pellet area ends by engaging the centrifuge chamber in a vortex mode, in which the centrifuge chamber is rotated and/or moved about the axis in such a way as to cause pelleted material to be dislodged and separated in resuspension fluid present in the tapered pellet area ends.

Resuspended material in the tapered pellet area ends 280/440 can be transferred to the center portion, i.e., to the center extraction well 270 in the bottom surface of the bottom portion 200 following resuspension. In some embodiments, transfer of the resuspended material involves engaging the centrifuge chamber 100 in a transfer mode, in which the centrifuge chamber 100 is rotated and/or moved about the axis 270 in such a way as to cause resuspended material to travel from the tapered pellet area ends down into the central portion of the container, where it settles in the center extraction well 270 of the bottom portion 200. For example, in some embodiments, the transfer mode involves causing the centrifuge chamber to decelerate rapidly, or alternatively abruptly. In some embodiments, the rapid deceleration is achieved via a braking system in the centrifuge.

Resuspended material located in the center extraction well 270 of the bottom portion 200 can be removed, in some embodiments, using the fluid transfer assembly 270. In some embodiments, a vacuum is used to pull the resuspended material up through the interior 670 of the cylindrically shaped inner straw, where it can be removed through the inner straw port 640.

### Kits

Embodiments provided herein also relate to kits that include the centrifuge chambers discussed herein above. For example, some embodiments relate to a kit that consists of, or comprises, a container as described herein. The kits can also optionally include a fluid transfer assembly as described herein, one or more syringes configured to couple to a port of the centrifuge chamber, or both. Some kits optionally include conduits capable of being coupled e.g., in a manner to provide a sterile fluid pathway, to the inner straw and outer straw ports of a fluid transfer assembly, and/or to one or more ports located in the top portion of the container, thereby providing a sterile fluid pathway/closed system.

The above description and figures are illustrative of preferred embodiments which achieve the objects, features, and advantages of the present invention, and it is not intended that the present invention be limited thereto.

1. A centrifuge chamber, comprising:
a container forming an interior chamber configured to receive a cellular suspension and having an exterior surface, said container comprising:
a bilaterally symmetrical bottom portion with an interior chamber and an exterior outer surface with symmetrical left and right sides; comprising
a center extraction well comprising a center point located in the center of the bottom portion, the center extraction well being configured to receive a first end of a fluid transfer assembly and to hold a portion of the cellular suspension or a desired cell population, wherein the center point defines an axis around which the centrifuge container rotates;
the left and right sides each having a central portion adjoined to a respective tapered pellet area end portion, wherein each central portion comprises a bottom surface and sidewalls, wherein the bottom surface of each central portion is sloped at a first angle θ1 less than 90° relative to the axis, the bottom surface sloping towards the center point such that the lowest point of each bottom surface of the central portion is the center point, the highest point of each bottom surface of the central portions being distal to the center point, the highest point being the location where each central portions adjoins the respective tapered pellet area end potion,
the tapered pellet area end portions each comprising sidewalls and a bottom surface, said tapered pellet area end bottom surfaces being sloped at a second angle θ2 greater than 90° relative to the axis, the lowest point of each bottom surface of the tapered pellet area end portions being distal to the center point, and the highest point of each tapered pellet area end bottom surface being the location where each tapered pellet area end portion adjoins the highest point of the respective central portion,
wherein the sidewalls of the central portions and respective tapered pellet area end portions comprise a top that forms a first mating surface;
a bilaterally symmetrical top portion comprising:
a left side and a right side, each side having a central portion adjoined to a respective tapered pellet area end portion;
sidewalls forming a second mating surface having the same shape and complementary angles to the first mating surface of the bottom portion;
a solid top surface with a center point aperture configured to accommodate the fluid transfer assembly, the center point defining the axis around which the centrifuge chamber rotates;
wherein the bottom portion and the top portion together form the interior chamber;
a means for locking the centrifuge chamber into a cradle of centrifuge comprising a motor.

2-29. (canceled)
